(19) United States
(12) Patent Application Publication
(10) Pub. No.: US 2009/0171264 A1
(43) Pub. Date: Jul. 2, 2009

(54) Medical Devices Based On Poly(Vinyl Alcohol)

(75) Inventors: Richard King, Warsaw, IN (US); Mark Hanes, Winona Lake, IN (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: DePuy Products, Warsaw, IN (US)

(21) Appl. No.: 12/338,324

(22) Filed: Dec. 18, 2008

Related U.S. Application Data
(60) Provisional application No. 61/015,806, filed on Dec. 21, 2007.

(51) Int. Cl. A61N 1/30; A61F 2/28; B29C 35/08; A61K 47/32

(52) U.S. Cl. 604/20; 424/423; 264/496; 514/772.2

(57) ABSTRACT

Provided are orthopedic implants and scaffolds comprising poly(vinyl alcohol) which has a degree of hydrolysis of at least 90% and a weight average molecular weight of at least 50,000. Also provided are methods for making same.

### RELATED APPLICATIONS

This application claims benefit of U.S. Application No. 61/015,806, filed Dec. 21, 2007, the disclosure of which is incorporated herein in its entirety.

### FIELD OF THE INVENTION

The present invention concerns, inter alia., medical devices based on poly(vinyl alcohol) and methods for making and using same.

### BACKGROUND OF THE INVENTION

Most long-term orthopedic implants contain synthetic hydrophobic polymers. Some metallic implants, for example, have an articulating surface made of a hydrophobic polymer such as ultra high molecular weight polyethylene. Wear particles from such hydrophobic polymers often induce adverse immune responses such as osteolysis. Furthermore, these polymers, while being bioinert, are not ideally suited for use as a cell scaffold or soft tissue replacement. Thus, there is a need in the art for an implant material that is more bio-friendly either in bulk form or porous construct.

### SUMMARY OF THE INVENTION

In some aspects, the invention relates to implants comprising poly(vinyl alcohol) (PVA), wherein said poly(vinyl alcohol) has a degree of hydrolysis of at least 90% and a weight-average molecular weight of at least 50,000. Some implants further comprise a therapeutic composition. The degree of hydrolysis is at least 95 or 98% in certain embodiments. Some preferred PVAs are cross-linked.

Some embodiments concern orthopedic implants. Orthopedic implants of the invention include those having an articulating surface that comprises poly(vinyl alcohol). Some implants can contain additional materials such as water, a plasticizer such as glycerol, or therapeutic compositions.

In some aspects, the invention concerns scaffolds for soft tissue repair and regeneration comprising the poly(vinyl alcohol) compositions described herein.

Other aspects of the invention concern methods forming articles comprising the PVA compositions described herein. One such method comprises

contacting poly(vinyl alcohol) having a weight average molecular weight of at least 50,000 and a degree of hydrolysis of at least 90% with an amount of one or more plasticizers that constitute 10-50% of the weight percent of the poly(vinyl alcohol), thereby forming a plasticized material; and

molding the plasticized material to form a consolidated article.

In some embodiments, the process concerns hydrating the consolidated glycerol-containing PVA article to a full water-saturation state.

In certain embodiments, the method further comprises increasing the Shore D hardness by subjecting said article to a temperature of or below 0° C. or −80° C. and then subjecting said article to a pressure below atmospheric pressure. If an decrease of Shore D hardness is desired, the article comprising cross-linked PVA can be contacted with an aqueous solution at a temperature of 70° C. to 95° C.

In some embodiments, the poly(vinyl alcohol) is in granular form when contacted with the glycerol. Suitable plasticizers include polyhydric alcohols such as glycerols. The plasticizers should have suitable thermal properties to be compatible with processing conditions.

Any suitable consolidation method can be used to form the articles. Such methods include compression molding and ram extrusion.

The methods can further comprise cross-linking the poly(vinyl alcohol) to form a cross-linked article.

Cross-linking can occur by any method known in the art. In some embodiments, the cross-linking is accomplished by exposing the poly(vinyl alcohol) to high-energy ionization radiation.

Some implants and scaffolds can be porous. Certain methods for making such articles use compression moldable materials which further comprise sodium chloride. In some methods, where the cross-linked article is contacted with water for a time and under conditions that are effective to remove at least a portion of the glycerol and sodium chloride. In some preferred embodiments, at least 90% of the glycerol and at least 90% of the sodium chloride are removed by contacting the cross-linked article with water.

The invention also concerns iontophoresis devices comprising a chamber comprising poly(vinyl alcohol), wherein said poly(vinyl alcohol) has a degree of hydrolysis of at least 90% and a weight average molecular weight of at least 50,000 Daltons; a therapeutic composition within said chamber; and an electrical power source in communication with said chamber. In some embodiments, the therapeutic composition is delivered transdermally. In some embodiments, the therapeutic agent has a positive or negative charge.

### BRIEF DESCRIPTION OF THE DRAWINGS

FIG. 1 shows a micrograph of porous water-saturated PVA of Example 3.

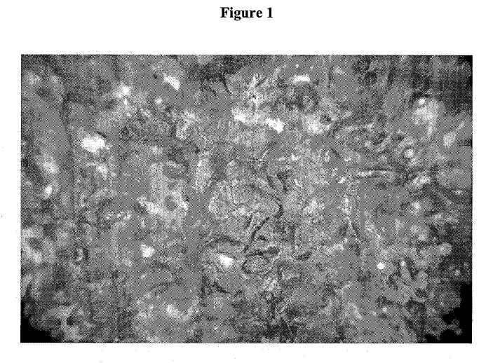

FIG. 2 shows a micrograph of porous water-saturated PVA of Example 3.

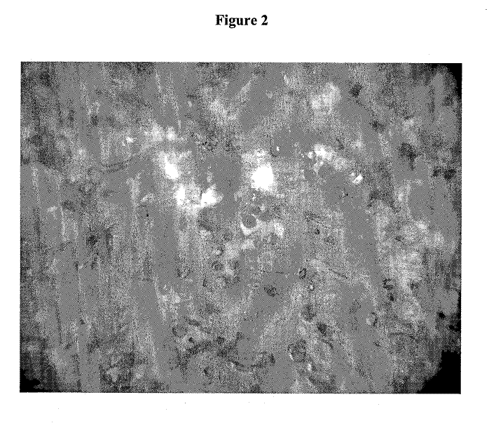

FIG. 3 presents a schematic for process relating to glyercol-plasticization of PVA resin.

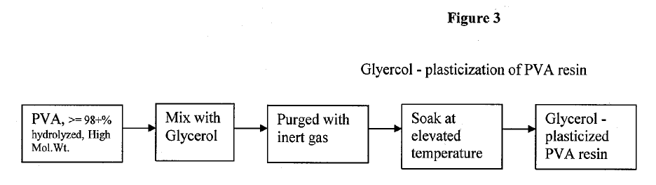

FIG. 4 presents a schematic for processes relating to fabricate various non-crosslinked PVA implant materials.

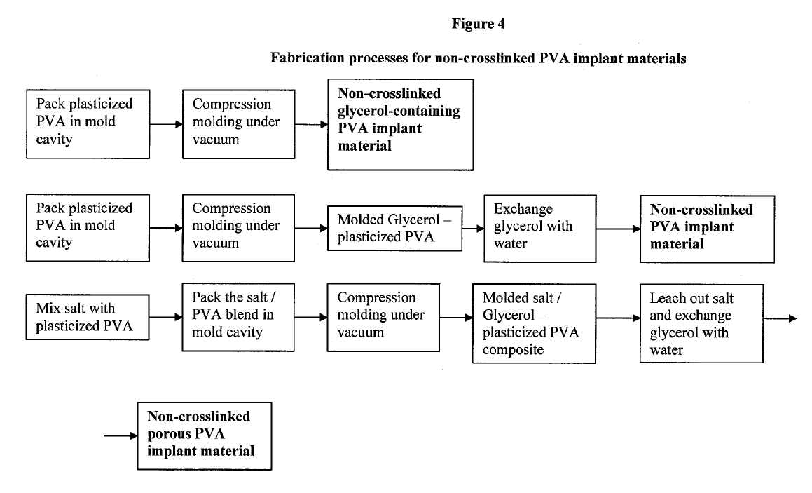

FIG. 5 presents a schematic for processes relating to fabricate various crosslinked PVA implant materials.

### DETAILED DESCRIPTION OF ILLUSTRATIVE EMBODIMENTS

The invention generally concerns implants comprising poly(vinyl alcohol), wherein said poly(vinyl alcohol) has a degree of hydrolysis of at least 90% and a weight-average molecular weight of at least 50,000 Daltons. Some implants additionally contain a therapeutic composition. Such implant can be placed in an animal (human, for example) body and release the therapeutic composition over time. Such procedures are well known to those skilled in the art.

In one aspect, the invention concerns hydrophilic orthopedic implants based on poly(vinyl alcohol) (PVA). These implants, unlike those made from hydrophobic polymers, are also useful as cell scaffolds or soft tissue replacement. Poly(vinyl alcohol) is more bio-friendly than the polymer used to make traditional implants.

In some embodiments, the articles of the invention contain 10 to 50 weight percent of water. In other embodiments, the articles contain 30% by weight or less of water.

One advantage of the invention is that the PVA structures of the invention are structurally stronger than those of conventional PVA hydrogels. Some structures have a Shore D hardness of at least 35.

Poly(vinyl alcohol) can be a fully hydrolyzed PVA, with all repeating groups being —CH₂—CH(OH)—, or a partially hydrolyzed PVA with varying proportions (1% to 25%) of pendant ester groups. PVA with pendant ester groups have repeating groups of the structure —CH₂—CH(OR)— where R is COCH₃ group or longer alkyls, as long as the desired properties are preserved. In some embodiments, the PVA preferably has a degree of hydrolysis of at least 98%. In certain embodiments, the PVA has a molecular weight of at least 100,000 Daltons (Mw).

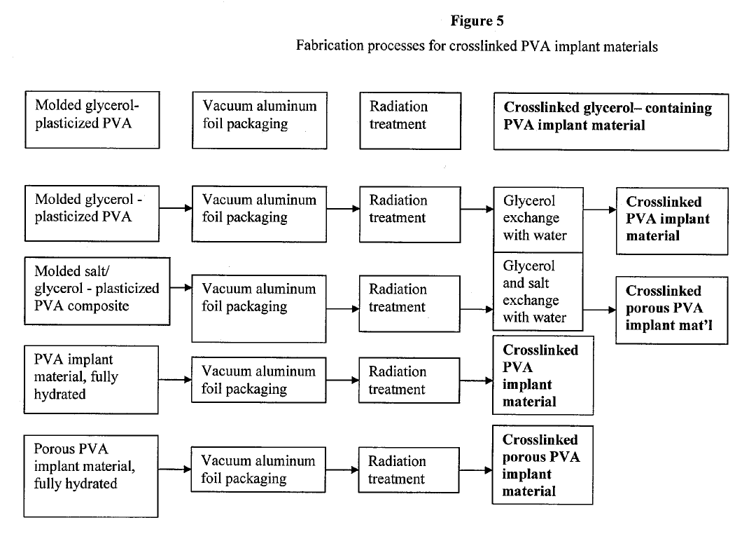

PVA is preferably cross-linked. Cross-linking of PVA can be accomplished, for example, by high-energy ionization radiation such as gamma radiation. One such scheme is presented in FIG. 5. In the alternative, chemical cross-linking can also be utilized.

The hardness of an article of the invention can be adjusted by subjecting the article to one or more freeze dry cycles. For example, the article can be subjected to a temperature of below 0° C., or −20° C., or −50° C., or −80° C. in the freeze cycle. The article can be subjected to the freezing temperatures from a few minutes to several hours. For example, 5 minutes to 24 hours. The drying cycle can be accomplished at a pressure below atmospheric pressure. For example, the pressure can be at or below 10⁻², 10⁻⁴, or 10⁻⁶ torr. The drying cycle can be performed at a variety of temperatures—below 0° C. in some embodiments. One or more freeze/dry cycles can increase the Shore D hardness. In some embodiments, the Shore D hardness is increased by at least 2, or 5, or 10 units.

The hardness can also be adjusted by soaking the article in water at a temperature above 70° C. In some embodiments, the article is soaked at a temperature above 80° C., or 90° C. The article can be subjected to the soaking from a few minutes to several hours. For example, 5 minutes to 24 hours. In some embodiments, the Shore D hardness is decreased by at least 2, or 5, 10 or 20 units.

As used herein, the term “hardness” refers to indentation hardness of non-metallic materials in the form of a flat slab or button as measured with a durometer. The durometer has a spring-loaded indenter that applies an indentation load to the slab, thus sensing its hardness. The hardness can indirectly reflect upon other material properties, such as tensile modulus, resilience, plasticity, compression resistance, and elasticity. Standard tests for material hardness include ASTM D2240. Unless otherwise specified, material hardness reported herein is in Shore D.

The articles (implants and scaffolds) of the invention can be vacuum foil packaged. Such techniques are known to those skilled in the art. These techniques include a process known as Gamma Vacuum Foil (GVF), as disclosed in U.S. Pat. No. 5,577,368 to Hamilton, et al.

Poly(vinyl alcohol) has high melting point and is generally known to degrade before it melts. In one aspect, the present invention provides a novel compression molding process that allows preparation of PVA components by plasticizing PVA resin with glycerol prior to compression molding. Plasticization process can be performed, for example, by soaking PVA resin in glycerol. In some embodiments, the soaking is performed by first soaking the PVA resin at room temperature, followed by a heat soak at a temperature above 70° C. (above 80° C., in some embodiments) for four hours or longer to produce a plasticized PVA resin. The plasticized PVA resin can then be consolidated at temperature between 350° F. (176.7° C.) and 420° F. (215° C.) with adequate pressurization.

As used herein, a plasticizer is a composition, that when added to PVA, increases the flexibility, workability, or moldability to the PVA.

Some embodiments include the use of compression molding to form articles such as implants. Compression molding techniques are known to those skilled in the art. In some preferred embodiments, an oxygen-reduced environment is preferred for plastization and/or compression molding. Suitable oxygen-reduced environments include reduced pressure, nitrogen or argon atmospheres, or combinations thereof.

Glycerol, a biocompatible lubricant, can be used as a part of the orthopedic implants. Alternatively, glycerol in PVA component can be exchanged with water by prolonged soaking in water or saline. This latter step allows production of a PVA component containing water or saline, rather than glycerol, within the PVA resin. Some embodiments can utilize plasticizing agents other than glycerol. In certain embodiments, other polyhydic alcohols are utilized.

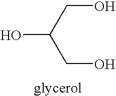

By “scaffolding”, it is meant a supporting matrix in which tissue can grow in a predetermined shape. This shape is predetermined by the shape of the scaffolding. The scaffold functions to support and shape the regenerated tissue. The manufacture of scaffolds is well known in the art.

By “implant” it is meant an article (such as a graft, device, scaffold, or joint replacement component) that is suitable for implantation in tissue. Implant devices are well known in the art. Joints that can benefit from the invention include, but are not limited to knees, ankles, shoulders, elbows, and wrists.

As used herein, the terms “water-saturated” and “fully hydrated” are considered equivalent.

A therapeutic agent may also be covalently attached to or contained in the implant or scaffold. The therapeutic agent is attached either chemically or enzymatically. The therapeutic agent may be attached without further modification or it may be conjugated with a spacer arm. If a spacer arm is used, the spacer arm may have a site that allows for cleavage of the spacer arm under discreet biological conditions. Upon cleavage of the spacer arm, the biological agents would then be free to diffuse from the implant or scaffold. A therapeutic drug that is compatible with the PVA material can be used.

Suitable therapeutic agents include one or more of the following: chemotactic agents; antibiotics, steroidal and non-steroidal analgesics; anti-inflammatories; anti-rejection agents such as immunosuppressants and anti-cancer drugs; various proteins (e.g. short chain peptides, bone morphogenic proteins, glycoprotein and lipoprotein); cell attachment mediators; biologically active ligands; integrin binding sequence; ligands; various growth and/or differentiation agents (e.g. epidermal growth factor, IGF-I, IGF-II, TGF-beta, growth and differentiation factors, fibroblast growth factors, platelet derived growth factors, insulin like growth factor, parathyroid hormone, parathyroid hormone related peptide, BMP-2; BMP-4; BMP-6; BMP-7; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin; heparan sulfate; DNA fragments and DNA plasmids. If other such substances have therapeutic value in the orthopaedic field, it is anticipated that at least some of these substances will have use in concepts of the present disclosure, and such substances should be included in the meaning of “therapeutic agents” unless expressly limited otherwise.

In some embodiments, the devices of the invention are iontophoresis devices. These devices allow a therapeutic agent to be administered to a patient in a non-invasive manner. In some embodiments, the agent is transdermally administered using repulsive electromotive force. Such force can use a small electrical charge that is applied to an iontophoretic chamber constructed using the PVA materials described herein. Iontophoresis devices contain at least two electrodes. Typically, both electrodes are positioned to be in intimate electrical contact with some portion of the skin of the body. One electrode, functioning as or associated with a chamber, contains the therapeutic agent which is to be delivered. The second electrode functions to complete the electrical circuit through the body. The chamber can contain a therapeutic agent that has the same charge as the chamber. For example, a positively charged chamber can be used to emit a positively charged agent from the device. Likewise, a negatively charged chamber can be utilized with a negatively charged agent. In some embodiment, the agent is a water soluble agent. Some therapeutic agents are local anesthetics such as lidocaine hydrochloride and fentanyl hydrochloride. See, for example, Parkinson, et at, Drug Delivery Technology, Vol. 7, No. 4, pages 54-60 (April 2007).

In contrast to traditional transdermal patches, the delivery of agents from an iontophoresis device can be controlled by control of the current applied to the device. In addition to control of the electrical current applied to the device, drug delivery is also impacted by the pH of the skin, the concentration of the agent in the device, agent characteristics such as charge, charge concentration, and molecular weight, and the skin resistance of a particular patient.

Some iontophoretic devices for delivery of a therapeutic agent having a positive or negative charge, comprise (i) a reservoir comprised of a poly(vinyl alcohol) polymer and containing a positively or negatively charged therapeutic agent and a counter ion, and (ii) an electrically conductive member comprising a material that is readily oxidizable to form a charged ionic species when the conductive member is in contact with the reservoir and a positive or negative voltage is applied to the reservoir. In some embodiments, when the reservoir comprising PVA is hydrated, it is permeable to the therapeutic agent.

Iontophoresis devices are well known to those skilled in the art. See, for example, U.S. Pat. Nos. 3,991,755; 4,141,359; 4,398,545; 4,250,878 and 5,711,761, whose disclosure related to iontophoresis devices and their uses incorporated by reference herein. Commercial iontophoresis devices include those produced by ALZA (IONSYS®) and IOMED. Typically, these devices utilize a battery-powered microprocessor DC current dose controller which is placed at the treatment site and connected to an electrode which is placed nearby on the patient's body. Some devices are a skin patch having a disposable low-voltage battery built into the device.

The invention is illustrated by the following examples that are intended to be illustrative and not limiting.

### EXAMPLES

### Example 1

### Cross-Linked PVA Implant Material

15.0 grams of PVA (99+% hydrolysis, 166,000 Dalton Mw) was mixed with 4.5 ml of glycerol and the mixture was allowed to soak for 24 hours. The mixture was then heat soaked at 80° C. for 8 hours. The resulting plasticized PVA resin was transferred to a 3.5″-diameter, 3-piece mold for consolidation. The PVA resin was heated to 420° F. (215.5° C.) at a heat up rate of 5-10° F./min. and consolidated under 1,000 psi pressure for 10 minutes, followed by cooling at a rate of 10-15° F./min. The resulting PVA plaque was packaged in a vacuum aluminum foil pouch for 50 KGy gamma radiation treatment.

Tensile data for glycerol-containing PVA versus cross-linked, glycerol-containing PVA is presented in Table 1. Tensile tests were performed per ASTM D 638 using Type V test specimens:

TABLE 1
Tensile data for glycerol-containing PVA versus
cross-linked, glycerol-containing PVA

| | Tensile | | Yield | | Break |
|---|---|---|---|---|---|
| | Modulus, | Strain at | Stress, | % Stain at | Stress, |
| | ksi | Yield, % | ksi | Break | ksi |
| PVA | 157 | 24 | 4.0 | 305 | 5.4 |
| Cross-linked | 88 | 6.7 | 1.9 | 463 | 6.0 |
| PVA | | | | | |

In the presence of glycerol, PVA crosslinks to form a network structure when exposed to gamma radiation. There is significant improvement in overall tensile property after radiation crosslinking. Interestingly, crosslinking boosts energy to break from 47 in-lb to 69 in-lb, a significant improvement in toughness and structural integrity.

### Example 2

### Water Saturated Crosslinked PVA Implant Material

30.0 grams of PVA (99+% hydrolysis, Mw=166,000 Daltons) was mixed with 9 ml of glycerol and the mixture was allowed to soak overnight. The mixture was then heat soaked at 194° F. (90° C.) for 6 hours. The resulting plasticized PVA was then transferred to 3-piece mold for consolidation. Consolidation was performed at 400° F. (204.4° C.) under 1200 psi for 10 minutes. (heat-up rate: 5-10° F./min. and cool-down rate: 10-15° F./min.) The resulting molded plaque was vacuum packaged in an aluminum foil pouch. The plaque was then treated with 75 KGy gamma radiation. The molded plaque was then soaked in distilled water for two days to replace glycerol.

Compression tests were run using the following method. Five disc test specimens (0.50″ Diameter×˜0.19″ Height) were compression loaded between parallel plates on a MTS Insight 5 tester at a crosshead speed of 0.4″/min. Tests were stopped when compression loads exceeded 95% of load cell rating (950 Lb). None of the test specimens failed in compression mode.

Double notched Izod impact tests were preformed using the following procedures. Five rectangular test specimen (0.25″×0.50″×2.5″) were notched and tested based on ASTM F 648. This test was used to assess toughness of the water saturated polyvinyl alcohol in comparison with one of the toughest polymers, ultra-high molecular weight polyethylene. Test results showed that the water saturated cross-linked polyvinyl alcohol is comparable to ultrahigh molecular weight polyurethane (UHMWPE) in terms of impact strength.

Table 2 presents compression properties and impact resistance for water saturated cross-linked PVA samples

TABLE 2
Compression properties and impact resistance
for water-saturated PVA samples.

| | |
|---|---|
| Compression stress | >4,800 psi (without fracture) |
| Compressive modulus | 16 ksi |
| Strain | >29% (without fracture) |
| Double notched Izod impact strength | 107 KJ/m² |

In the wet form, crosslinked PVA is pliable and has high compression strength and impact resistance.

### Example 3

### Macro-Porous PVA

20.0 gram of PVA (99+% hydrolysis, 146,000 Mw) was mixed with 6.0 ml of glycerol and allowed to soak overnight. The mixture was then heat soaked at 105° C. for 6 hours to produce a plasticized PVA mixture. 10.0 grams of table salt was then mixed with the plasticized PVA resin using a Turbula mixer. Consolidation of the resulting mixture was performed using the molding cycle described in Example 2. The molded article was soaked in water for extended period of 5 days to leach out salt and to exchange glycerol with water. Table 3 shows characteristics of the porous water-saturated PVA (Tensile tests were performed according ASTM D638, Type V test specimen).

TABLE 3
Characteristics of the porous water-saturated PVA

| | |
|---|---|
| Water content, % of total weight | 23.4% |
| Tensile strength at break | 292 psi |

### Example 4

### Freeze-Dried PVA Material

20.0 gram of PVA (99+% hydrolysis, M_{w}=166,000 Dalton) was mixed with 6 ml of glycerol and the mixture was allowed to soak overnight. The mixture was then heat soaked at 110° C. for four hours. The resulting plasticized PVA was then transferred to 3.5″ D 3-piece mold for consolidation. Consolidation was performed at 380° F. under 600 psi pressure for 5 minutes (heat-up rate: 5-10° F./min. and cool-down rate: 10-15° F./min.)

This non-crosslinked PVA material was then soaked in water at room temperature for two days to replace glycerol with water. Hardness for the glycerol-plasticized PVA was 62 (Shore D) and the water-saturated PVA had water content of 34.5% (water weight/PVA weight) and hardness of 38 (Shore D).

This water-saturated PVA block was further processed by going through a cycle of freezing drying, overnight freezing at −80° C. and drying at 40×10⁻⁶ torr for six hours. The freeze-dried PVA had hardness of 46 (Shore D).

### Example 5

### Crosslinked PVA of Reduced Crystallinity

40.0 gram of PVA (99+% hydrolysis, M_{w}=166,000 Dalton) was mixed with 12 ml of glycerol and the mixture was allowed to soak overnight. The mixture was then heat soaked at 176° F. (80° C.) for six hours. The resulting plasticized PVA was then transferred to 3.5″ D 3-piece mold for consolidation. Consolidation was performed using two-soak stage process: at 220° F. (104.4° C.) under 1040 psi for 5 minutes and at 400° F. (204.4° C.) under 1560 psi for 15 minutes (heat-up rate: 5-10° F./min. and cool-down rate: 10-15° F./min.) The resulting molded plaque was vacuum packaged in an aluminum foil pouch and gamma irradiated for 50 KGy.

The crosslinked PVA material contained 17.3% glycerol (glycerol weight per PVA weight) due to in-process loss and to a less extent glycerol bleeding from PVA. This material was relatively rigid, having hardness of 66 (Shore D). The crosslinked PVA material was then soaked in 80° C. water for two hours. The hot water soaking process removed glycerol and dissolved non-crosslinked PVA. It significantly softened the crosslinked PVA. The reconstituted PVA had water content of 34.4% (water weight per PVA weight) and hardness of 36 (Shore D). The water-saturated, crosslinked PVA block then went through a cycle of freeze drying, overnight freezing at −80° C. and drying at 40×10⁻⁶ torr for six hours. The freeze-dried PVA block had hardness of 42 (Shore D).

What is claimed:

1. A medical device comprising:
poly(vinyl alcohol), wherein said poly(vinyl alcohol) has a degree of hydrolysis of at least 90% and a weight average molecular weight of at least 50,000 Daltons, and
a therapeutic composition;
said device having 10-50 weight percent content of at least one of water and plasticizer.

2. The medical device of claim 1, wherein the poly(vinyl alcohol) is cross-linked.

3. The medical device of claim 1, wherein the poly(vinyl alcohol) is at least 98% hydrolysed.

4. The medical device of claim 1, further comprising a plasticizer.

5. The medical device of claim 4, wherein the plasticizer comprises glycerol.

6. The medical device of claim 1, further comprising water.

7. The medical device of claim 1, wherein said medical device is an orthopedic implant.

8. The medical device of claim 7 having an articulating surface that comprises said poly(vinyl alcohol).

9. The medical device of claim 7, further comprising a therapeutic composition.

10. The medical device of claim 7, further comprising water.

11. The medical device of claim 1, wherein said medical device is a scaffold for soft tissue repair and regeneration

12. The scaffold of claim 11, further comprising a therapeutic composition.

13. A method of forming an article comprising:
contacting poly(vinyl alcohol) having a weight average molecular weight of at least 50,000 Daltons and a degree of hydrolysis of at least 90% with an amount of one or more plasticizers that constitutes 10-50% of the weight percent of the poly(vinyl alcohol), thereby forming a plasticized material; and
molding the plasticized material to form a consolidated article.

14. The method of clam 13, wherein the plasticizer comprises glycerol.

15. The method of claim 14, further comprising cross-linking the poly(vinyl alcohol) to form a cross-linked article.

16. The method of claim 15, wherein the cross-linking is accomplished by exposing the poly(vinyl alcohol) to high-energy ionization radiation.

17. The method of claim 15, further comprising contacting the cross-linked article with water for a time and under conditions that are effective to remove at least a portion of the glycerol.

18. The method of claim 13, wherein the poly(vinyl alcohol) is at least 98% hydrolysed.

19. The method of claim 13, further comprising altering the hardness of said article by
(a) subjecting said article to a temperature below 0° C. and then subjecting said article to a pressure below atmospheric pressure; or
(b) cross-linking the poly(vinyl alcohol) to form cross-linked PVA and subjecting said article comprising cross-linked PVA to an aqueous solution at a temperature above 70° C.

20. The method of claim 14, wherein the compression moldable material further comprises sodium chloride.

21. The method of claim 20, further comprising contacting the cross-linked article with water for a time and under conditions that are effective to remove at least a portion of the glycerol and sodium chloride.

22. The method of claim 21, wherein at least 90% of the glycerol and at least 90% of the sodium chloride are removed by contacting the cross-linked article with water.

23. The method of claim 14, wherein the poly(vinyl alcohol) is in granular form when contacted with the glycerol.

24. The method of claim 13, wherein the cross-linked article is an orthopedic implant.

25. The method of claim 13, wherein the cross-linked article is a scaffold for soft tissue regeneration.

26. An iontophoresis device comprising:
a chamber comprising poly(vinyl alcohol), wherein said poly(vinyl alcohol) has a degree of hydrolysis of at least 90% and a weight average molecular weight of at least 50,000 Daltons;
a therapeutic composition within said chamber; and
an electrical power source in communication with said chamber.

27. The iontophoresis device of claim 26, wherein the poly(vinly alcohol) is cross-linked.

28. The iontophoresis device of claim 26, wherein the poly(vinyl alcohol) is at least 98% hydrolysed.

29. The iontophoresis device of claim 26, wherein said therapeutic composition is delivered transdermally.

30. The iontophoresis device of claim 26, wherein said therapeutic agent has a positive or negative charge
